(19) United States
(12) Patent Application Publication
(10) Pub. No.: US 2025/0347704 A1
(43) Pub. Date: Nov. 13, 2025

(54) Phosphorylated Neurofilament Heavy Chain Protein as a Biomarker of Blast-Induced Traumatic Brain Injury

(71) Applicant: The Government of the United States, as Represented by the Director of the Defense Health Agency, Fort Detrick, MD (US)

(72) Inventors: Peethambaran Arun, Clarksburg, MD (US); Joseph B. Long, Clarksville, MD (US)

(21) Appl. No.: 19/192,445

(22) Filed: Apr. 29, 2025

Related U.S. Application Data
(60) Provisional application No. 63/644,563, filed on May 9, 2024.

(51) Int. Cl. G01N 33/68

(52) U.S. Cl. CPC G01N 33/6896; G01N 2440/14; G01N 2800/28; G01N 2800/52

(57) ABSTRACT

A method for the detection and assessment of traumatic brain injury includes measuring a level or amount of phosphorylated neurofilament heavy chain protein (pNFH) in at least one plasma sample from a mammal that has been exposed to a blast; and comparing the measured level or amount of pNFH in the at least one plasma sample to a level or amount of pNFH in at least one plasma sample of a mammal that has not been exposed to a blast.

This US patent application claims priority of U.S. Ser. No. 63/644,563 filed in the US Patent and Trademark Office on May 9, 2024, the entirety of which is incorporated herein by reference.

### BACKGROUND OF INVENTION

Blast-induced traumatic brain injury (bTBI) is a significant concern for both military and civilian populations in response to the increased use of improvised explosive devices. Identifying biomarkers that could aid in the proper diagnosis and assessment of both acute and chronic bTBI is an urgent need since little progress has been made towards this goal. Addressing this knowledge gap is especially important in military veterans who are receiving assessment and care often years after their last blast exposure.

Neuron-specific phosphorylated neurofilament heavy chain protein (pNFH) has been evaluated as a reliable biomarker of different neurological disorders, as well as brain trauma resulting from contact sports and acute stages of brain injury of different origin other than blast exposure.

Further, it is known that pNFH can be measured in the cerebrospinal fluid (CSF) of rats and that the CSF levels of pNFH increase after blast exposure. However, no studies have shown any correlation of pNFH levels in rat plasma with neurobehavioral changes (acute and chronic) after blast exposure.

A disadvantage with the use of pNFH in CSF as a possible biomarker of bTBI is that CSF must be collected for analysis, which is inconvenient and is an invasive potentially painful procedure. Thus, there is a need to find an alternative way to analyze pNFH as a biomarker for bTBI.

### SUMMARY OF INVENTION

The invention provides in a first embodiment a method for the detection and/or assessment of traumatic brain injury comprising measuring a level or amount of pNFH in at least one plasma sample from a mammal that has been exposed to a blast and comparing the measured level or amount of pNFH in the at least one plasma sample to a level or amount of pNFH in at least one plasma sample of a mammal that has not been exposed to a blast. The mammal may have a blast-induced traumatic brain injury.

The invention provides in a second embodiment further to any of the previous embodiments a method for the detection and assessment of traumatic brain injury in which the mammal is a ferret, cat, dog, pig, sheep, or monkey.

The invention provides in a third embodiment further to any of the previous embodiments a method for the detection and assessment of blast-induced traumatic brain injury in which the mammal is a human.

The invention provides in a fourth embodiment further to any of the previous embodiments a method for the detection and assessment of traumatic brain injury in which at least one plasma sample is obtained about 2 to about 4 hours after a blast exposure.

The invention provides in a fifth embodiment further to any of the previous embodiments a method for the detection and assessment of traumatic brain injury in which at least one plasma sample is obtained about 24 hours after a blast exposure.

The invention provides in a sixth embodiment further to any of the previous embodiments a method for the detection and assessment of traumatic brain injury further comprising diagnosing bTBI in the mammal based on measured levels of pNFH in a plurality of plasma samples over a period of time.

The invention provides in a seventh embodiment further to any of the previous embodiments a method for the detection and/or assessment of traumatic brain injury further comprising providing a prognosis for the mammal based on correlating changes in neurological behavior of the mammal exposed to a blast with measured levels of pNFH in a plurality of plasma samples over a period of time.

The invention provides in an eighth embodiment further to any of the previous embodiments a method for the detection and/or assessment of traumatic brain injury further comprising treating the mammal exposed to the blast by administering a pharmaceutically effective amount of a composition or drug after the blast exposure and monitoring the efficacy of the treatment by measuring plasma levels of pNFH.

### BRIEF DESCRIPTION OF THE DRAWINGS

FIG. 1 is a graph showing the changes in pNFH levels in plasma samples of ferrets at different time intervals (4 hours, 24 hours, 1 week, 1 month) after a blast exposure compared to pNFH levels in control (sham) plasma samples for ferrets that were not exposed to a blast.

FIGS. 2A-2D are bar graphs showing changes in horizontal activity count of ferrets at different time intervals (1 day (FIG. 2A), 7 days (FIG. 2B), 14 days (FIG. 2C), and 28 days (FIG. 2D)) after a blast exposure compared to a control (sham) of ferrets that were not exposed to a blast.

FIGS. 3A-3D are the same as FIGS. 2A-2D, but include data for blast-treated ferrets (ferrets treated with a pharmaceutical agent 30 min. after the blast).

FIGS. 4A-4B are bar graphs showing changes in discrimination index of ferrets at different time intervals (2 days (FIG. 4A) and 28 days (FIG. 4B)) after a blast exposure compared to a control (sham) of ferrets that were not exposed to a blast.

FIGS. 5A-5B are the same as FIGS. 4A-4B, but include data for blast-treated ferrets.

FIGS. 6A-6D are bar graphs showing changes in sleep fragmentation index of ferrets at different time intervals (1 day (FIG. 6A), 7 days (FIG. 6B), 14 days (FIG. 6C), and 28 days (FIG. 6D)) after a blast exposure compared to a control (sham) of ferrets that were not exposed to a blast.

FIGS. 7A-7D are the same as FIGS. 6A-6D, but include data for blast-treated ferrets.

### DETAILED DESCRIPTION OF INVENTION

The present invention is directed to a method of measuring pNFH levels in at least one plasma sample of a mammal exposed to blast and comparing the measured pNFH levels in the at least one plasma sample to pNFH levels in at least one plasma sample of a mammal that has not been exposed to a blast. In a specific embodiment, the pNFH level in a plasma sample may be measured by ELISA.

In this detailed description, references to “one embodiment”, “an embodiment”, or “in embodiments” mean that the feature being referred to is included in at least one embodiment of the invention. Moreover, separate references to “one embodiment”, “an embodiment”, or “embodiments” do not necessarily refer to the same embodiment; however, neither are such embodiments mutually exclusive, unless so stated, and except as will be readily apparent to those skilled in the art. Thus, the invention can include any variety of combinations and/or integrations of the embodiments described herein.

As used herein “substantially”, “generally”, “about”, and other words of degree are relative modifiers intended to indicate permissible variation from the characteristic so modified (e.g., ±0.1%, ±0.5%, ±1.0%, ±2%, ±5%, ±10%, ±20%). It is not intended to be limited to the absolute value or characteristic which it modifies but rather possessing more of the physical or functional characteristic than its opposite, and preferably, approaching or approximating such a physical or functional characteristic.

According to an embodiment of the present invention, pNFH may be detected in the plasma of mammals. In a specific embodiment, pNFH can be measured in the plasma of ferrets. Ferrets have a gyrencephalic brain that is comparable to a human brain unlike rats, which have a lissencephalic brain. The present invention is also directed to measuring pNFH in the plasma of other mammals including, but not limited to, dogs, cats, pigs, sheep, monkeys, and humans.

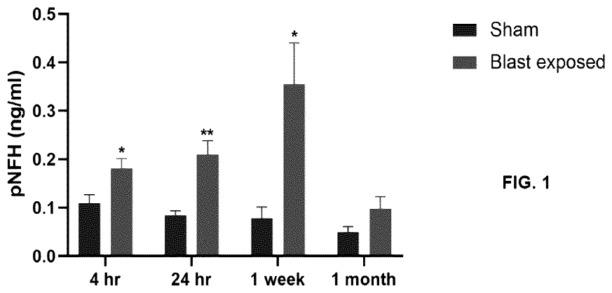

A level of pNFH in a plasma sample, measured for example in ng/ml, increases after a blast exposure. As shown in FIG. 1, in one embodiment, a pNFH level may be elevated in the plasma of a ferret from about 4 hours, about 24 hours, about 1 week, and about 1 month after a blast exposure as compared to a sham control (ferrets that have not been exposed to a blast). The shown values are expressed as mean±SEM. The number of measurements of pNFH level were four (n=4) for 4 hours and 1 week and eight (n=8) for 24 hours and 1 month. Statistical analysis was carried out using Student t test (*p<0.05; p**<0.01).

According to the present invention, a level of pNFH in the plasma may be measured periodically over a set period of time.

According to one embodiment of the present invention, based on the comparing of a measurement of pNFH in at least one plasma sample, for example a plurality of plasma samples, to a measurement of pNFH in at least one plasma sample of a control, a mammal may be diagnosed with bTBI.

In another embodiment, a prognosis for a mammal having been subjected to a blast-induced traumatic brain injury may be provided by correlating pNFH measurements in a plurality of plasma samples over a period of time to observed changes in at least one neurological behavior of the mammal. Neurofilament proteins are present only in the neurons. Therefore, whatever changes occur in the levels of pNFH in plasma also reflect changes happening to neurons in the brain.

A change in at least one neurological behavior may include, but is not limited to anxiety, depression or depression-like symptoms, sleep changes, and the like.

In one specific embodiment, a change in at least one neurological behavior of the mammal exposed to a blast begins at day 1, with a maximal observed change at about day 14, and a return towards normal at day 28.

According to the present invention, mammals exposed to a blast may be treated by administering a pharmaceutically effective amount of a pharmaceutical composition or drug after blast exposure and monitoring the efficacy of a treatment by measuring levels of pNFH in a plurality of plasma samples.

In embodiments, the treatment may include administering at least one pharmaceutically effective amount or dose of an inhibitor of at least one toll-like receptor (TLR), for example, two or more TLRs, or multiple TLRs; an anti-inflammatory peptide, P13 (available from 13Therapeutics of Portland, OR); or a synthetic form of PreImplantation factor (a peptide secreted by the embryos during early embryonic development and pregnancy). In embodiments, the administration may be one or more doses of a pharmaceutically effective amount of pharmaceutical composition administered orally or by injection (e.g., peritoneal injection). In embodiments, the dosage may be about 2 mg/kg.

The following are non-limiting examples of the present invention.

### Examples

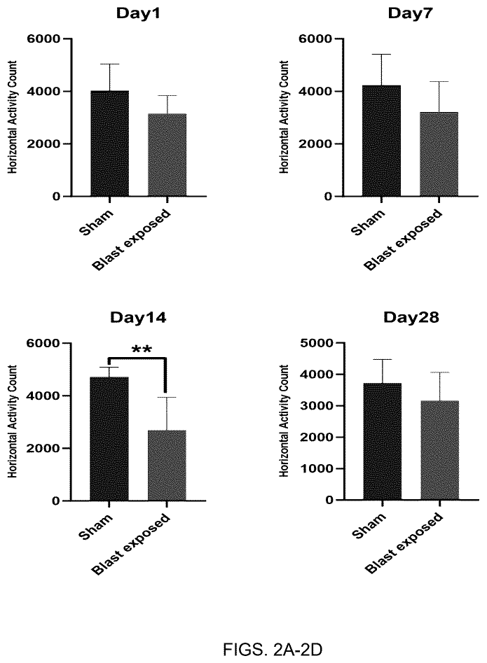

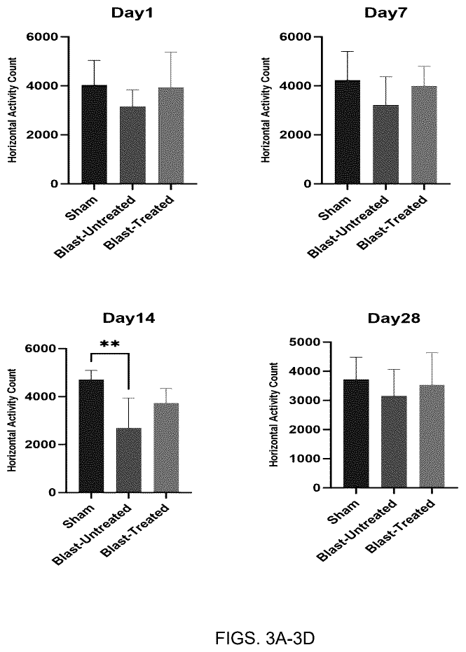

As shown in FIGS. 2A-2D, a change in at least one neurological behavior includes a change in horizontal activity count. Horizontal activity (movement) count was measured in an open field locomotion apparatus. Decreased horizontal activity indicated anxiety and/or depression or depression-like behavior. The horizontal activity count decreased in ferrets at 1 day (FIG. 2A), 7 days (FIG. 2B), 14 days (FIG. 2C), and 28 days (FIG. 2D) after a blast exposure as compared to a sham control. Values (time in seconds) are expressed as mean±SEM. The number of measurements of horizontal activity were n=10 for each group at each point in time. Statistical analysis was carried out using Analysis of Variance (*p<0.01). The horizontal activity deficit was statistically significant (**) at day 14. FIGS. 3A-3D are the same FIGS. 2A-2D, but include data from ferrets that were exposed to a blast and were subsequently (30 min. after blast) treated with P13.

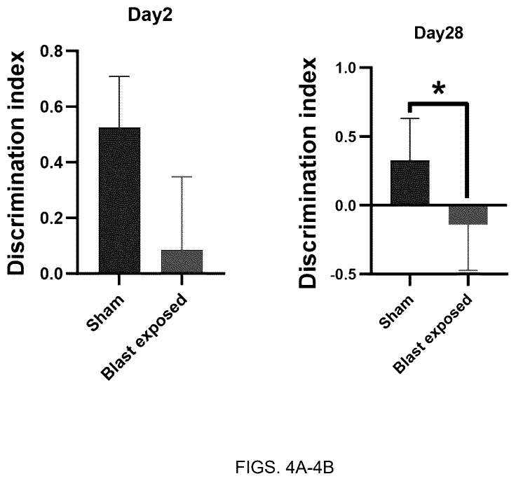

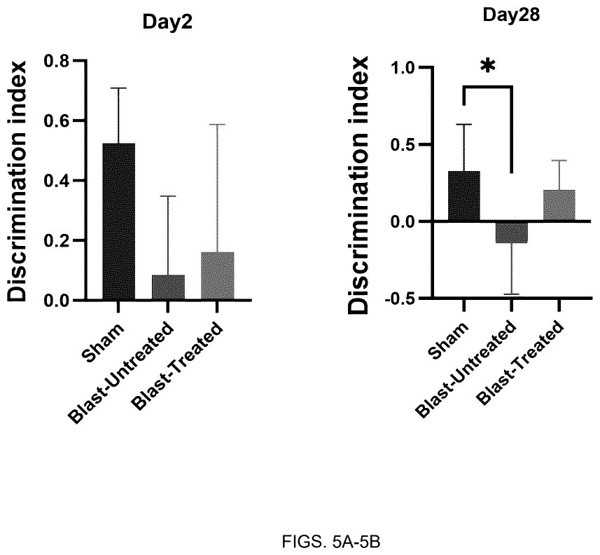

As shown in FIGS. 4A-4B, a change in at least one neurological behavior includes a change in a discrimination index. A discrimination index was measured using a novel object recognition test for assessing short-term memory function. Decreased discrimination abilities indicated a decrease in memory. A discrimination index decreased in ferrets from 2 days (FIG. 4A) to 28 days (FIG. 4B) after a blast exposure as compared to a sham control. Values of the index are expressed as mean±SEM. The number of measurements of discrimination were six to eight (n=6 to 8) for each group at each point in time. Statistical analysis was carried out using Analysis of Variance (*p<0.05). The decrease in discrimination index was statistically significant (*) at day 28. FIGS. 5A-5B are the same a FIGS. 4A-4B, but include data from ferrets that were exposed to a blast and were subsequently treated with P13.

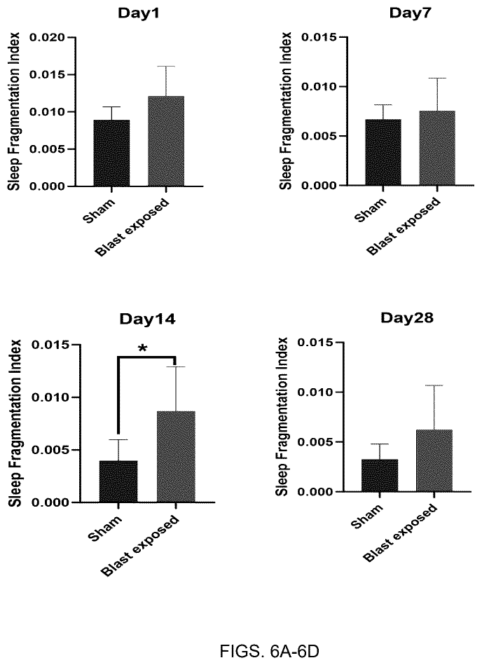

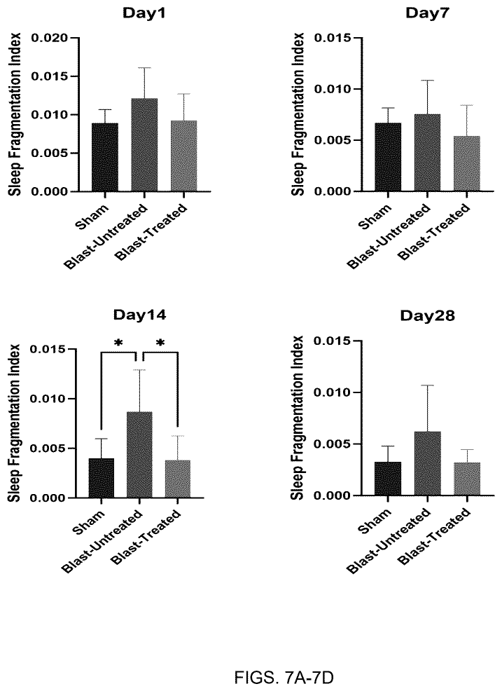

As shown in FIGS. 6A-6D, a change in at least one neurological behavior includes a change in sleep fragmentation (sleep-wake cycles) of ferrets at different time intervals (1 day (FIG. 6A), 7 days (FIG. 6B), 14 days (FIG. 6C), and 28 days (FIG. 6D)) after a blast exposure compared to a control (sham) of ferrets that were not exposed to a blast. A sleep fragmentation index was measured using actigraphy. Increased sleep fragmentation indicated disturbed sleep. A sleep fragmentation index increased in ferrets after blast exposure and a statistically significant increase was observed at day 14 as compared to the sham control. Values of the index are expressed as mean±SEM. The number of measurements of sleep fragmentation was six to eight (n=6 to 8) for each group at each point in time. Statistical analysis was carried out using Analysis of Variance (*p<0.05). FIGS. 7A-7D are the same as FIGS. 6A-6D, but include data from ferrets that were exposed to a blast and were subsequently treated with P13.

Although the present invention has been described in terms of particular exemplary and alternative embodiments, it is not limited to those embodiments.

Alternative embodiments, examples, and modifications which would still be encompassed by the invention may be made by those skilled in the art, particularly in light of the foregoing teachings.

Those skilled in the art will appreciate that various adaptations and modifications of the exemplary and alternative embodiments described above can be configured without departing from the scope and spirit of the invention. Therefore, it is to be understood that, within the scope of the appended claims, the invention may be practiced other than as specifically described herein.

What is claimed is:

1. A method for the detection and assessment of traumatic brain injury comprising:
measuring a level or amount of phosphorylated neurofilament heavy chain protein (pNFH) in at least one plasma sample from a mammal that has been exposed to a blast; and
comparing the measured level or amount of pNFH in the at least one plasma sample to a level or amount of pNFH in at least one plasma sample of a mammal that has not been exposed to a blast.

2. The method according to claim 1, wherein the mammal has blast-induced traumatic brain injury (bTBI).

3. The method according to claim 1, wherein the mammal is a ferret.

4. The method according to claim 1, wherein the mammal is a cat, dog, pig, sheep or monkey.

5. The method according to claim 1, wherein the mammal is a human.

6. The method according to claim 1, wherein the at least one plasma sample is obtained about 2 hours to about 4 hours after a blast exposure.

7. The method according to claim 1, wherein the at least one plasma sample is obtained about 24 hours after a blast exposure.

8. The method according to claim 1, wherein the at least one plasma sample is obtained about 7 days to about 1 month after a blast exposure.

9. A method according to claim 1, further comprising diagnosing bTBI in the mammal based on measured levels of pNFH in a plurality of plasma samples over a period of time.

10. A method according to claim 1, further comprising providing a prognosis for the mammal based on correlating changes in at least one neurological behavior of the mammal exposed to the blast with measured levels of pNFH in a plurality of plasma samples over a period of time.

11. A method according to claim 10, wherein the at least one neurological behavior comprises anxiety, depression or depression-like symptoms.

12. A method according to claim 10, wherein a change in at least one neurological behavior comprises a decrease in short-term memory function.

13. A method according to claim 10, wherein a change in at least one neurological behavior comprises an increase in sleep fragmentation.

14. A method according to claim 1, further comprising treating the mammal exposed to the blast by:
administering a pharmaceutically effective amount of a composition or drug after blast exposure; and
monitoring the efficacy of a treatment by measuring levels of pNFH in a plurality of plasma samples.

15. The method according to claim 14, wherein the levels of pNFH are measured from about 2 days to about 1 month after the blast exposure.

16. The method according to claim 14, wherein the composition or drug comprises an inhibitor of at least one toll-like receptor.

17. The method according to claim 14, wherein the composition or drug comprises an inhibitor of two or more toll-like receptors.

18. The method according to claim 14, wherein the composition or drug comprises an inhibitor of a plurality of toll-like receptors.

19. The method according to claim 14, wherein the composition or drug comprises a peptide.
